(19) United States
(12) Patent Application Publication
(10) Pub. No.: US 2015/0259642 A1
(43) Pub. Date: Sep. 17, 2015

(54) METHODS AND COMPOSITIONS FOR DEGRADING OIL SLUDGE

(71) Applicant: INDIAN INSTITUTE OF TECHNOLOGY MADRAS, Chennai (IN)

(72) Inventors: Jitendra SANGWAI, Dist Buldhana (IN); Mukesh DOBLE, Ashok Nagar (IN); Sakthipriva NALLUSAMY, Trichy (IN)

(21) Appl. No.: 14/639,574

(22) Filed: Mar. 5, 2015

(30) Foreign Application Priority Data
Mar. 6, 2014 (IN) 1150/CHE/2014

(51) Int. Cl. C12N 1/20; C09K 8/52

(52) U.S. Cl. CPC C12N 1/20; C09K 8/52

(57) ABSTRACT

Methods and systems for degrading oil sludge are disclosed. In one embodiment, a method of degrading an oil sludge in a pipeline may involve introducing a microbial mixture comprising a Pseudomonas sp. and a nutrient medium into the pipeline such that the microbial mixture contacts the oil sludge. In some embodiments, the Pseudomonas sp. may be Pseudomonas aeruginosa, Pseudomonas fluorescens, or any combination thereof. In a further embodiment, a method of biodegrading an oil sludge may involve contacting the oil sludge with a microbial mixture comprising a Pseudomonas sp. and a nutrient medium, wherein about 70% to about 99% of the oil sludge is degraded in a period of about 1 day to about 3 days.

### RELATED APPLICATION

This application claims priority benefit under Title 35 §119(a) of Indian Patent Application No. 1150/CHE/2014, filed Mar. 6, 2014, entitled, “Methods and Compositions for Degrading Oil Sludge,” the contents of which are herein incorporated by reference.

### BACKGROUND

Crude oil is indispensable for the world's economy and industrial growth as it is the primary fuel source for combustion engines and a raw material for many chemical products including pharmaceuticals, solvents, fertilizers, pesticides, and plastics. Some of the operational and technical problems encountered by the petroleum industry include corrosion, scale deposition, emulsion formation, and wax and sludge deposition in production tubings and pipelines. These problems play a role in both upstream and downstream industries, resulting in the loss of billions of dollars per year. It is, therefore, crucial to overcome these problems with economically feasible and quick solutions.

Crude oil typically contains higher hydrocarbons such as paraffins (waxes), along with lower percentages of aromatics, resins, and asphaltenes. The waxes are mainly in a dissolved state at reservoir conditions due to high temperature and/or pressure. As the waxy crude oil flows from the reservoir to the surface, the reduction in the system pressure and temperature causes the waxes to separate from the bulk flowing stream and deposit as waxy crystals. This phenomenon occurs in well heads, pumps, tubes, pipelines, and in long distance pipelines that transport crude oil from oilfields to refineries due to the reduction of crude oil temperature to or below the wax appearance temperature (WAT). Several methods are employed to remove the deposited wax, which mainly include chemical, thermal, and mechanical methods. Chemical methods include use of solvents, dispersants, surfactants, and wax crystal modifiers. However, these are expensive and potentially toxic.

In addition to wax deposition, build-up of tank bottom sludge (TBS) in oil storage facilities imposes a serious problem to the petroleum industry. TBS is a gradual accumulation of waxes in the lower portion of the petroleum storage tanks. Over time, as more and more sludge is deposited and settles, the TBS may become thick, resulting in a compacted, dense layer of sludge. Such accumulation can render the tank unusable as the compacted sludge may result in an inability to suck and dispatch crude oil for delivery through pipeline. Expensive chemicals and emulsion techniques are routinely used to clear TBS. Thus, there is a need to develop methods to degrade oil sludge in pipelines and storage tanks ideally using economical and environmentally friendly methods.

### SUMMARY

Disclosed herein are methods and compositions to degrade an oil sludge. In one embodiment, a method of degrading an oil sludge in a pipeline may involve introducing a microbial mixture comprising a Pseudomonas sp. and a nutrient medium into the pipeline such that the microbial mixture contacts the oil sludge. In some embodiments, the Pseudomonas sp. may be Pseudomonas aeruginosa, Pseudomonas fluorescens, or any combination thereof.

In another embodiment, a pipeline may comprise an oil sludge, a microbial mixture comprising a Pseudomonas sp. and a nutrient medium disposed within the pipeline, wherein the microbial mixture is in contact with the oil sludge.

In an additional embodiment, a biofilm may comprise a Pseudomonas sp. and a nutrient medium, wherein the nutrient medium comprises KH₂PO₄, Na₂HPO₄, NH₄Cl, NaCl, glucose, MgSO₄, or any combination thereof.

In a further embodiment, a kit to degrade an oil sludge may include a microbial mixture comprising a Pseudomonas sp. and a nutrient medium, and instructions for contacting the microbial mixture with the oil sludge under conditions to at least partly degrade the oil sludge.

In an additional embodiment, an article may comprise at least one surface for contacting an oil sludge, and a biofilm coating on the at least one surface, wherein the biofilm coating comprises a Pseudomonas sp.

In a further embodiment, a method of biodegrading an oil sludge may involve contacting the oil sludge with a microbial mixture comprising a Pseudomonas sp. and a nutrient medium, wherein about 70% to about 99% of the oil sludge is degraded in a period of about 1 day to about 3 days.

In yet another embodiment, a method of preventing a build-up of an oil sludge in a pipeline may involve coating an inner surface of the pipeline at least partly with a biofilm comprising a Pseudomonas sp. and a nutrient medium, wherein the biofilm degrades the oil sludge upon contact with the oil sludge.

In a further embodiment, a method of preventing a build-up of an oil sludge in a pipeline may involve providing an oil flow in the pipeline, and introducing a microbial mixture comprising a Pseudomonas sp. and a nutrient medium into the pipeline.

In an additional embodiment, a method of pretreating crude oil may involve contacting crude oil with a microbial mixture comprising a Pseudomonas sp. and a nutrient medium, wherein the microbial mixture degrades about 70% to about 99% of an oil sludge present in the crude oil in a period of about 1 day to about 3 days.

### BRIEF DESCRIPTION OF THE FIGURES

FIG. 1 illustrates a biofilm formation and degradation of wax in an oil pipeline according to an embodiment.

FIGS. 2A-D show the growth characteristics of Pseudomonas sp. in the presence of n-eicosane and n-hexadecane, according to embodiments. FIG. 2A shows colony forming unit and biomass weight measurements of Pseudomonas aeruginosa; FIG. 2B shows the growth rate of Pseudomonas aeruginosa; FIG. 2C shows colony forming unit and biomass weight measurements of Pseudomonas fluorescens; FIG. 2D shows the growth rate of Pseudomonas fluorescens.

FIGS. 3A-B show emulsification activity and surface tension of culture broth of Pseudomonas aeruginosa (FIG. 3A) and Pseudomonas fluorescens (FIG. 3B) grown in the presence of n-eicosane and n-hexadecane, according to embodiments.

FIGS. 4A-B show viscosity measurements of the culture broth before and after degradation of n-hexadecane (FIG. 4A) and n-eicosane (FIG. 4B) by Pseudomonas aeruginosa and Pseudomonas fluorescens, according to some embodiments.

FIGS. 5A-B show the rate of degradation of n-hexadecane and n-eicosane by Pseudomonas aeruginosa (FIG. 5A), and Pseudomonas fluorescens (FIG. 5B), according to an embodiment.

### DETAILED DESCRIPTION

This disclosure is not limited to the particular systems, devices and methods described, as these may vary. The terminology used in the description is for the purpose of describing the particular versions or embodiments only, and is not intended to limit the scope.

Disclosed herein are methods for and compositions useful for degrading oil sludge by microbial treatment. Non-limiting examples of an oil sludge include petroleum sludge, hydraulic oil, asphaltene, wax, a C₁₅-C₁₀₀ aliphatic, a paraffin, an aromatic, an olefin, a resin, tank bottom sludge, power plant waste, paint, an electroplating waste, or any combination thereof. In addition, oil sludge may also include drill cuttings, shipping sludge, oil spills on water and/or soil, grease, hydraulic oil, sludge cake from biotreatment plants, power plants, chemical plants, and the like. Typically, the hydrocarbons in oil sludge include combinations of both aliphatics (C₁₅-C₁₀₀) and aromatics (C₆-C₄₀). Examples of hydrocarbons found in such sludge include pentachlorophenols (PCPs), polychlorinated biphenyls (PCBs), polyaromatic hydrocarbons (PAHs) such as naphthalene, anthracene, acenapthene, acenaphthylene, and pyrene, polynuclear aromatics (PNAs), 2,4,6-trinitrotoluene (TNT), nitrocellulose (NC), benzene, toluene, ethylbenzene, xylene (BTEX), olefins, paraffins, isoparaffins, xenobiotics, and the like. The oil sludge described herein may be in various containers such as a wellbore tubing, an oil pipeline, or a storage tank. The oil sludge may also be an oil spill, an oil-water emulsion, crude oil, soil mixed with oil, or any combination thereof.

In some embodiments, a method of biodegrading an oil sludge involves contacting the oil sludge with a microbial mixture comprising a Pseudomonas sp. and a nutrient medium. In some embodiments, the Pseudomonas sp. used herein may be Pseudomonas aeruginosa, Pseudomonas fluorescens, or any combination thereof. In addition, other microbes that may be used are Acinetobacter species, Brevibacillus brevis, Bacillus cereus, Bacillus licheniformis, Bacillus mojavensis, Bacillus thermoleovorans, Enterobacter, Geobacillus kaustophilus, Geobacillus thermodenitrificans Gordonia amicalis, Rhodococcus, Pseudomonas putida, Pseudomonas syringae, Pseudomonas protegens, and any combination thereof. In some embodiments, a single Pseudomonas sp., such as Pseudomonas aeruginosa may be used. In other embodiments, Pseudomonas fluorescens alone may be used.

In some embodiments, the nutrient medium may contain salts, such as KH₂PO₄, Na₂HPO₄, NH₄Cl, NaCl, MgSO₄, or any combination thereof. In some embodiments, the nutrient medium may also contain a carbon source, such as glucose, fructose, maltose, starch, yeast extract, and the like. For example, the nutrient medium may contain about 0.1 wt. % to about 0.4 wt. %, about 0.1 wt. % to about 0.3 wt. %, or about 0.1 wt. % to about 0.2 wt. % KH₂PO₄, and ranges between any two of these values. In other embodiments, the nutrient medium may contain about 0.3 wt. % to about 0.8 wt. %, about 0.3 wt. % to about 0.7 wt. %, about 0.3 wt. % to about 0.6 wt. %, or about 0.3 wt. % to about 0.5 wt. % Na₂HPO₄, and ranges between any two of these values. In additional embodiments, the nutrient medium may contain about 0.1 wt. % to about 0.3 wt. %, 0.1 wt. % to about 0.2 wt. %, or 0.1 wt. % to about 0.15 wt. % NH₄Cl, and ranges between any two of these values. In addition, the nutrient medium may also contain about 0.3 wt. % to about 0.7 wt. %, about 0.3 wt. % to about 0.6 wt. %, about 0.3 wt. % to about 0.5 wt. %, or about 0.3 wt. % to about 0.4 wt. % NaCl, and ranges between any two of these values. In certain embodiments, the nutrient medium may also contain about 0.01 wt. % to about 0.05 wt. %, about 0.01 wt. % to about 0.04 wt. %, about 0.01 wt. % to about 0.03 wt. %, or about 0.01 wt. % to about 0.02 wt. % MgSO₄, and ranges between any two of these values. In some embodiments, the nutrient medium may contain about 0.5 wt. % to about 1 wt. %, about 0.5 wt. % to about 0.9 wt. %, about 0.5 wt. % to about 0.8 wt. %, or about 0.5 wt. % to about 0.7 wt. % glucose, and ranges between any two of these values (including their endpoints). In some embodiments, the nutrient medium may contain any combination of salts and carbon source in any concentration described herein.

In some embodiments, the oil sludge is contacted with the microbial mixture at a temperature of about 5° C. to about 50° C., about 5° C. to about 40° C., about 5° C. to about 30° C., or about 5° C. to about 10° C. Specific examples include about 5° C., about 10° C., about 20° C., about 30° C., about 40° C., about 50° C., and ranges between any two of these values (including their endpoints).

In some embodiments, the microbes described herein are capable of forming a biofilm for contact with the oil sludge. In some embodiments, the biofilm may comprise a mixture of microbes along with nutrients described herein. Additionally, the sludge may be supplemented with additives and nutrients to further promote formation of the biofilm.

The microbes described herein may have hydrocarbon degrading genes making them genetically disposed for the degradation of oil sludge. In some embodiments, the microbial mixture described herein may degrade the oil sludge to a lower alkane, an ester, a primary alcohol, an aldehyde, a fatty acid, a dicarboxylic acid, acetyl CoA, or any combination thereof. In some embodiments, the microbial mixture may degrade about 70% to about 99% of the oil sludge, about 70% to about 90% of the oil sludge, about 70% to about 85% of the oil sludge, or about 70% to about 80% of the oil sludge in a period of about 1 day to about 3 days. Specific examples include about 70%, about 75%, about 80%, about 90%, about 95%, about 99%, and ranges between any two of these values (including their endpoints). For example, the microbial mixture may degrade the oil sludge about 80% in 1 day. In some embodiments, the microbial mixture may degrade the oil sludge about 80% in 1 day. In some embodiments, the microbial mixture may degrade the oil sludge about 90% in 1 day. In some embodiments, the microbial mixture may degrade the oil sludge about 80% in 2 days. In some embodiments, the microbial mixture may degrade the oil sludge about 80% in 3 days. In some embodiments, the microbial mixture may degrade the oil sludge about 90% in 2 days. In some embodiments, the microbial mixture may degrade the oil sludge about 99% in 1 day. In some embodiments, the microbial mixture may degrade the oil sludge about 99% in 2 days. In an ideal embodiment, the microbial mixture degrades the oil sludge about 100%.

In some embodiments, the microbial mixture may further contain at least one surfactant. The surfactant may be stable up to a temperature of about 60° C., about 70° C., about 80° C., about 90° C., or 100° C. In some embodiments, the surfactant may be stable at a pH of about 2 to about 12, about 2 to about 8, about 2 to about 7, or a pH of about 2 to about 5. Specific examples include about pH 2, about pH 4, about pH 6, about pH 8, about pH 10, about pH 12, and ranges between any two of these values (including their endpoints). In some embodiments, the Pseudomonas sp. in the microbial mixture may secrete the at least one surfactant. For example, Pseudomonas sp. may secrete a rhamnolipid surfactant.

In some embodiments, the surfactant present in the microbial mixture may help to emulsify the oil sludge, and decrease the viscosity of the oil sludge. In some embodiments, the microbial mixture may have an emulsification index of about 30% to about 70%, about 30% to about 60%, about 30% to about 50%, or about 30% to about 40%. Specific examples include about 30%, about 40%, about 50%, about 60%, about 70%, and ranges between any two of these values (including their endpoints). In addition, the microbial mixture when in contact with the oil sludge for a sufficient period of time may decrease the viscosity of the oil sludge by about 30% to about 60% at below wax appearance temperature (WAT). In some embodiments, the microbial mixture may decrease the viscosity by about 30% to about 50%, about 30% to about 40%, or about 30% to about 35%, and ranges between any two of these values (including their endpoints). In some embodiments, the viscosity of the oil sludge may be reduced sufficiently to permit flow of the oil sludge under special clean-out conditions. For example, the special clean-out conditions may include injection of pressurized gas (hydrocarbon gas, nitrogen, carbon dioxide, and the like) or circulation of steam around the closed system containing oil sludge. The pressurized gas may be injected at about 50 bar to about 200 bar, depending up on the type of gas being used. The temperature of steam may be about 150° C. to about 200° C. In some embodiments, the viscosity of the oil sludge may be reduced sufficiently to permit flow of the oil sludge under normal operating process conditions. For example, the viscosity of the oil sludge may be reduced to about 0.01 cP to about 5 cP.

The microbial mixture disclosed herein may be in contact with the oil sludge for various periods of time, without significant loss of its biodegrading function. For example, the microbial mixture may be in contact with the oil sludge for about 1 day to about 60 days, about 1 day to about 50 days, about 1 day to about 40 days, about 1 day to about 20 days, or about 1 day to about 10 days. Specific examples include about 1 day, about 2 days, about 3 days, about 5 days, about 10 days, about 20 days, about 30 days, about 40 days, about 50 days, about 60 days, and ranges between any two of these values (including their endpoints).

Also described herein are methods to degrade an oil sludge in a pipeline. Non-limiting examples of an oil sludge in the pipeline may be petroleum sludge, hydraulic oil, asphaltene, wax, a C₁₅-C₁₀₀ aliphatic, a paraffin, an aromatic, an olefin, a resin, or any combination thereof. The pipeline may be a part of a wellbore tubing, a well head, an oil storage tank, or any combination thereof. For example, the pipeline may be a part of a system stretching from an oil well to a storage facility, or it may be a part of a series of pipelines connecting a storage facility to a distribution facility. The pipeline may also be part of a petrochemical facility, a gas and oil refinery, a chemical plant, a crude oil distillation unit, and the like.

In some embodiments, a method of degrading an oil sludge in a pipeline involves introducing a microbial mixture comprising a Pseudomonas sp. and a nutrient medium into the pipeline such that the microbial mixture contacts the oil sludge. In some embodiments, the Pseudomonas sp. used herein may be Pseudomonas aeruginosa, Pseudomonas fluorescens, or any combination thereof. In some embodiments, the nutrient medium may contain salts, such as KH₂PO₄, Na₂HPO₄, NH₄Cl, NaCl, MgSO₄, or any combination thereof. In some embodiments, the nutrient medium may also contain a carbon source, such as glucose, fructose, maltose, starch, yeast extract, and the like. For example, the nutrient medium may contain about 0.1 wt. % to about 0.4 wt. %, about 0.1 wt. % to about 0.3 wt. %, or about 0.1 wt. % to about 0.2 wt. % KH₂PO₄, and ranges between any two of these values. In other embodiments, the nutrient medium may contain about 0.3 wt. % to about 0.8 wt. %, about 0.3 wt. % to about 0.7 wt. %, about 0.3 wt. % to about 0.6 wt. %, or about 0.3 wt. % to about 0.5 wt. % Na₂HPO₄, and ranges between any two of these values. In additional embodiments, the nutrient medium may contain about 0.1 wt. % to about 0.3 wt. %, 0.1 wt. % to about 0.2 wt. %, or 0.1 wt. % to about 0.15 wt. % NH₄Cl, and ranges between any two of these values. In addition, the nutrient medium may also contain about 0.3 wt. % to about 0.7 wt. %, about 0.3 wt. % to about 0.6 wt. %, about 0.3 wt. % to about 0.5 wt. %, or about 0.3 wt. % to about 0.4 wt. % NaCl, and ranges between any two of these values. In certain embodiments, the nutrient medium may also contain about 0.01 wt. % to about 0.05 wt. %, about 0.01 wt. % to about 0.04 wt. %, about 0.01 wt. % to about 0.03 wt. %, or about 0.01 wt. % to about 0.02 wt. % MgSO₄, and ranges between any two of these values. In some embodiments, the nutrient medium may contain about 0.5 wt. % to about 1 wt. %, about 0.5 wt. % to about 0.9 wt. %, about 0.5 wt. % to about 0.8 wt. %, or about 0.5 wt. % to about 0.7 wt. % glucose, and ranges between any two of these values (including their endpoints). In some embodiments, the nutrient medium may contain any combination of salts and carbon source in any concentration described herein.

In some embodiments, the microbial mixture contacts the oil sludge in the pipeline at a temperature of about 5° C. to about 50° C., about 5° C. to about 40° C., about 5° C. to about 30° C., or about 5° C. to about 10° C. Specific examples include about 5° C., about 10° C., about 20° C., about 30° C., about 40° C., about 50° C., and ranges between any two of these values (including their endpoints).

In some embodiments, the microbial mixture may form a biofilm on at least a part of an inside surface of the pipeline. The biofilm may form when microbes in the microbial mixture approach the surface of the pipeline. An electrical charge may build on the surface of the pipeline and may attract the microbes carrying an opposite charge. Within a short period of time, microbes in the growing biofilm may become firmly attached to the surface and to each other by means of tendrils or filaments. This attachment may be due to secretion of a polysaccharide material that entraps the microbes and debris within a glue-like matrix. The biofilm environment may also contain a rich layer of nutrients that is capable of supporting rapid growth of the microbes within the biofilm. Thus, due to formation of the biofilm, the microbes may effectively degrade the oil sludge.

The biofilm, with additional nutrients may be allowed to form on the inner surfaces of pipeline structures, prior to them being placed in the field. In this manner, a biofilm will be ready for use upon installation. The inclusion of nutrients allows for continued health of the biofilm.

The microbes described herein may have hydrocarbon degrading genes that genetically predispose them for the degradation of oil sludge. In some embodiments, the microbial mixture and/or the biofilm described herein may degrade the oil sludge in the pipeline to a lower alkane, an ester, a primary alcohol, an aldehyde, a fatty acid, a dicarboxylic acid, acetyl CoA, or any combination thereof. The microbial mixture when in contact with the oil sludge for a sufficient period of time may decrease the amount of oil sludge at least partly in the pipeline. In some embodiments, the microbial mixture may degrade about 70% to about 99% of the oil sludge, about 70% to about 90% of the oil sludge, about 70% to about 85% of the oil sludge, or about 70% to about 80% of the oil sludge in a period of about 1 day to about 3 days. Specific examples include about 70%, about 75%, about 80%, about 90%, about 95%, about 99%, and ranges between any two of these values (including their endpoints). In an ideal embodiment, the microbial mixture may degrade about 100% of the oil sludge.

In some embodiments, the microbial mixture and/or the biofilm may further contain at least one surfactant. The surfactant may be stable up to a temperature of about 60° C., about 70° C., about 80° C., about 90° C., or 100° C. In some embodiments, the surfactant may be stable at a pH of about 2 to about 12, about 2 to about 8, about 2 to about 7, or a pH of about 2 to about 5. Specific examples include about pH 2, about pH 4, about pH 6, about pH 8, about pH 10, about pH 12, and ranges between any two of these values (including their endpoints).

In some embodiments, the Pseudomonas sp. in the microbial mixture may secrete at least one surfactant, such as a rhamnolipid. The rhamnolipid secreted by the microbe may act as a biosurfactant and may have lower critical micelle concentration. When the wax crystalline particles are formed in the pipeline, the biosurfactant may form a micellar layer around them and keep them suspended in the flowing crude oil stream. Due to the lowering of interfacial tensional between the wax and the flowing crude oil, the wax or the oil sludge is solubilized and makes them transportable by reducing the viscosity.

In some embodiments, the surfactant present in the microbial mixture and/or the biofilm may help to emulsify the oil sludge, and decrease the viscosity of the oil sludge. In some embodiments, the microbial mixture may have an emulsification index of about 30% to about 70%, about 30% to about 60%, about 30% to about 50%, or about 30% to about 40%. Specific examples include about 30%, about 40%, about 50%, about 60%, about 70%, and ranges between any two of these values (including their endpoints). In addition, the microbial mixture when in contact with the oil sludge for a sufficient period of time may decrease the viscosity of the oil sludge by about 30% to about 60% at below wax appearance temperature (WAT). In some embodiments, the microbial mixture may decrease the viscosity by about 30% to about 50%, about 30% to about 40%, or about 30% to about 35%, and ranges between any two of these values (including their endpoints).

In certain embodiments, the microbial mixture and/or the biofilm is able to degrade the oil sludge in the presence of a light source, such as sunlight, or in an absence of a light source, such as darkness, or any combination thereof. For example, the microbial mixture is able to degrade the oil sludge present deep in an oil pipe and may not require sunlight and oxygen for its biological function.

The microbial mixture and/or the biofilm disclosed herein may be in contact with the oil sludge for various periods of time, without sufficient loss of its biodegrading function. For example, the microbial mixture may be in contact with the oil sludge for about 1 day to about 60 days, about 1 day to about 50 days, about 1 day to about 40 days, about 1 day to about 20 days, or about 1 day to about 10 days. Specific examples include about 1 day, about 2 days, about 3 days, about 5 days, about 10 days, about 20 days, about 30 days, about 40 days, about 50 days, about 60 days, and ranges between any two of these values (including their endpoints).

Also disclosed herein are methods to improve flow assurance issues using microbial treatment. Such methods allow not only for the reduction of existing oil sludge, but also for the reduction of oil sludge forming solids by treating them before they accumulate into an oil sludge. In some embodiments, the method may involve oil sludge degradation and/or degradation of oil sludge forming solids (waxes, paraffins, waxy crystals, and sludge cakes) with the help of thermotolerant microbes Pseudomonas aeruginosa and/or Pseudomonas fluorescens. The microbial mixture when in contact with the oil flow for a sufficient period of time may degrade any existing oil sludge while also degrading any oil sludge forming solids present in the oil flow and promote flow assurance in the pipeline. The microbes may be typically inserted at an injection port upstream to the location where the temperature in pipeline expected to fall below WAT.

A representative process is shown in FIG. 1 and generally includes: injecting a microbial mixture containing Pseudomonas sp. and nutrient medium into an oil wellbore tubing or an oil pipeline; optionally forming a biofilm containing microbes on the interior surface of the tubing; allowing the microbes to degrade paraffin/wax in the pipeline; and transporting oil through the tubing. Thus, the accumulation of deposited wax at susceptible areas of the tubing is reduced. Constriction caused by accumulation of oil sludge within the tubing is thereby deterred or prevented, and blockage of the flow of oil through the tubing is postponed or avoided.

Disclosed herein are embodiments, such as a pipeline having an oil sludge and a microbial mixture, wherein the microbial mixture includes a Pseudomonas sp. and a nutrient medium. The microbial mixture may be in contact with the oil sludge. The Pseudomonas sp. may be Pseudomonas aeruginosa, Pseudomonas fluorescens, or any combination thereof. In some embodiments, the nutrient medium may comprise KH₂PO₄, Na₂HPO₄, NH₄Cl, NaCl, glucose, MgSO₄, or any combination thereof. For example, the nutrient medium may comprise about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof. Non-limiting examples of an oil sludge in the pipeline may be petroleum sludge, hydraulic oil, asphaltene, wax, a C₁₅-C₁₀₀ aliphatic, a paraffin, an aromatic, an olefin, a resin, or any combination thereof. The microbial mixture described herein is configured to degrade the oil sludge to a lower alkane, an ester, a primary alcohol, an aldehyde, a fatty acid, a dicarboxylic acid, acetyl CoA, or any combination thereof. In some embodiments, the pipeline may further include a liquid crude oil.

In some embodiments, a kit to degrade an oil sludge may include a microbial mixture comprising a Pseudomonas sp. and a nutrient medium, and instructions for contacting the microbial mixture with the oil sludge under conditions to at least partly degrade the oil sludge.

In additional embodiments, an article may comprise at least one surface for contacting an oil sludge, and a biofilm coating on the at least one surface, wherein the biofilm coating comprises a Pseudomonas sp. In certain embodiments, the biofilm coating may further comprise a nutrient medium. Further, biofilm coating may be configured to degrade an oil sludge. Non-limiting examples of an article may be a pipe, wellbore tubing, an oil pipeline, or a storage tank.

Disclosed herein are methods to delay or reduce build-up of an oil sludge in a pipeline. In some embodiments, the method includes coating an inner surface of the pipeline, or a portion thereof, at least partly with a biofilm comprising a Pseudomonas sp. and a nutrient medium, wherein the biofilm degrades the oil sludge forming materials present in the oil flow. The Pseudomonas sp. may be Pseudomonas aeruginosa, Pseudomonas fluorescens, or any combination thereof. In other embodiments, the nutrient medium may contain about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof. Further, the biofilm may degrade about 70% to about 99% of the oil sludge to a lower alkane, an ester, a primary alcohol, an aldehyde, a fatty acid, a dicarboxylic acid, acetyl CoA, or any combination thereof in a period of about 1 day to about 3 days.

In other embodiments, a method to delay, reduce, or prevent a build-up of an oil sludge in a pipeline may involve providing an oil flow in the pipeline, and introducing a microbial mixture comprising a Pseudomonas sp. and a nutrient medium into the pipeline. The Pseudomonas sp. may be Pseudomonas aeruginosa, Pseudomonas fluorescens, or any combination thereof. In other embodiments, the nutrient medium may contain about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof. Further, the microbial mixture may degrade about 70% to about 99% of the oil sludge to a lower alkane, an ester, a primary alcohol, an aldehyde, a fatty acid, a dicarboxylic acid, acetyl CoA, or any combination thereof in a period of about 1 day to about 3 days.

Waxy crude oil can be pre-treated with microbes to reduce viscosity before transportation through pipelines. In yet another embodiment, a method of pretreating crude oil may involve contacting crude oil with a microbial mixture comprising a Pseudomonas sp. and a nutrient medium, wherein the microbial mixture degrades about 70% to about 99% of an oil sludge present in the crude oil in a period of about 1 day to about 3 days. In an ideal embodiment, the microbial mixture degrades about 100% of the oil sludge. The Pseudomonas sp. may be Pseudomonas aeruginosa, Pseudomonas fluorescens, or any combination thereof. In other embodiments, the nutrient medium may contain about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

Microbial mixtures disclosed herein may also be used to reduce wellbore skin damage. Injection of the microbial mixture in the wellbore may degrade the deposited wax near the wellbore and reduce the viscosity of the oil, making it easy to flow. In addition, the microbial mixture may also be used to remove tank bottom sludge. The microbial mixture may be injected into a storage tank containing existing tank bottom sludge. The microbes may degrade heavier hydrocarbons in the sludge to lower alkanes and fatty acids, and make the sludge flowable. Further, the microbial mixture described herein may also be used in de-emulsification of oil-water emulsion in oil and gas facilities. For example, the microbial mixture can be injected into a separator or a wellhead. Inside the separator, the microbes may break the oil-water emulsion and increase the efficiency of the separator. Further, these microbes can also be used to upgrade the heavy and waxy crude oil to lighter components. Such methods and techniques described herein may find application in refineries, chemical industries, fertilizer plants, distilleries, pharmaceuticals manufacturing plants, effluent treatment plants, and the like.

### EXAMPLES

### Example 1

### Growth of Pseudomonas aeruginosa and Pseudomonas fluorescens in Nutrient Medium

Pseudomonas aeruginosa was isolated from the petroleum sediments near Chennai, India. Pseudomonas fluorescens was isolated from ocean water near Ennore port, Chennai, India. A growth media was prepared by mixing solution ‘A’ and solution ‘B.’ Solution A contained 3 grams/liter potassium dihydrogen phosphate (KH₂PO₄), 6 grams/liter disodium hydrogen phosphate (Na₂HPO₄), 2 grams/liter ammonium chloride (NH₄Cl), and 5 grams/liter sodium chloride (NaCl). Solution B contained 8 grams/liter glucose and 0.1 gram/liter magnesium sulphate (MgSO₄.7H₂O). Both solutions A and B were sterilized by autoclaving at 120° C. for 2 hours. Equal volumes of sterilized solutions A and B were mixed in a flask (final volume 200 mL), and about 1 mL of Pseudomonas aeruginosa or Pseudomonas fluorescens were inoculated in separate flasks. About 0.1 gram of n-eicosane or 1 mL of n-hexadecane was also introduced in the flask and the microbial growth was monitored.

The flasks with the microbial culture were kept in an orbital shaker at 35° C., 180 rpm, for over a period of two months. For analyzing the growth of microbes, samples of culture broth were collected at regular intervals of 4 hours for the first 2 days. For the rest of the analysis, samples were collected at 10 day intervals. The growth of the microbes was determined by colony forming units (CFU) and biomass dry weight. CFU was determined by a spread plate method as follows. Culture media was serially diluted with saline solution (1:10) and an aliquot (0.1 ml) from each tube was spread on a petri dish containing agar medium. The petri dishes were incubated in an orbital shaker for 24 hours and the number of colonies was counted. Biomass dry weight was measured by obtaining culture broth (1 mL) at regular intervals (every 5 days). The culture broth was centrifuged at 1500 rpm for 30 minutes and the pellet obtained was dried overnight at 60° C., cooled, and weighed.

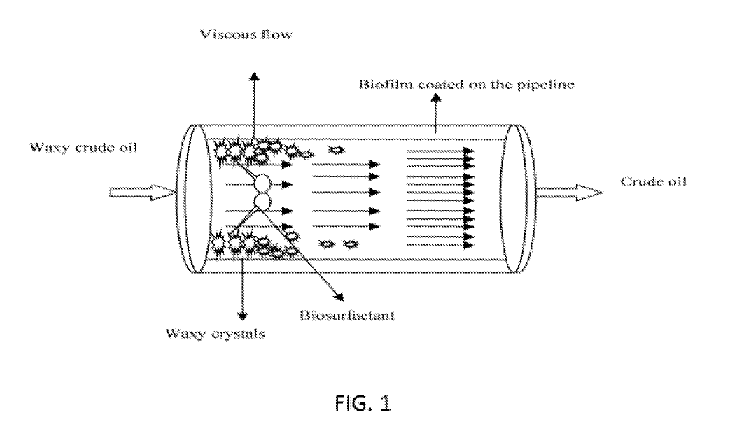

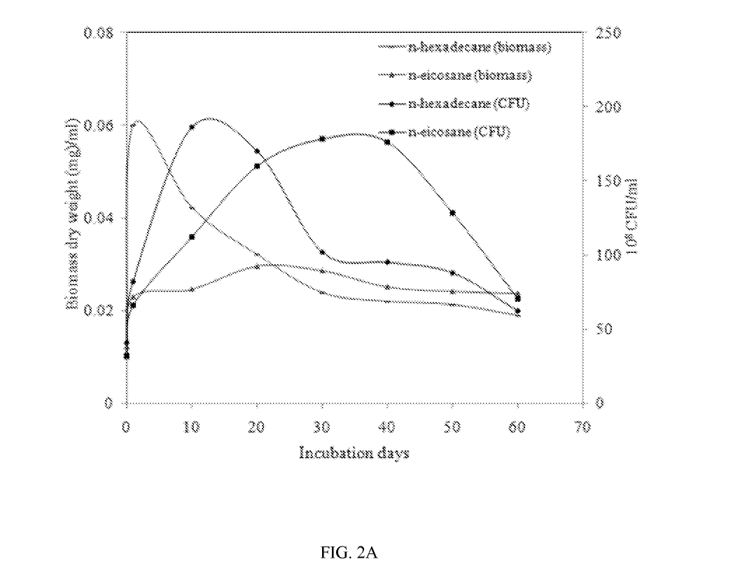

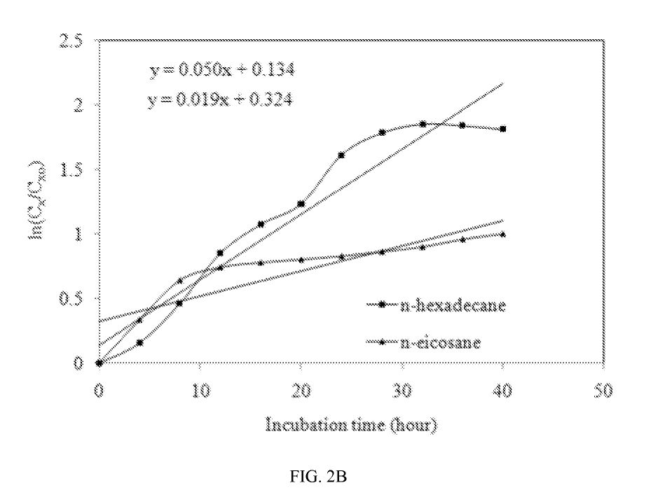

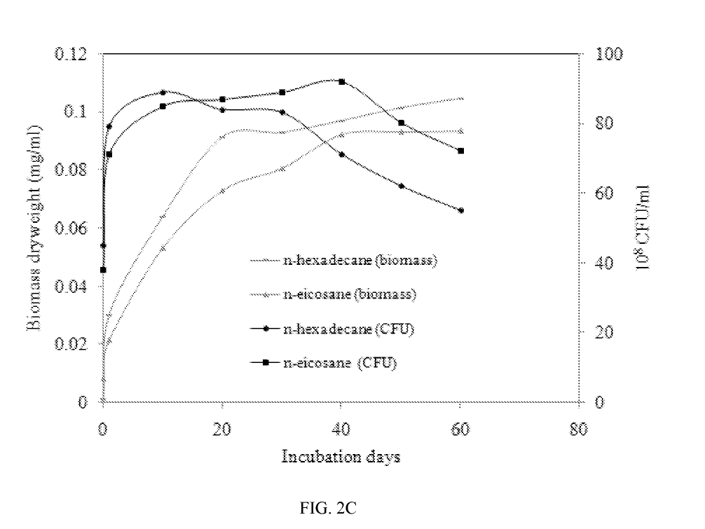

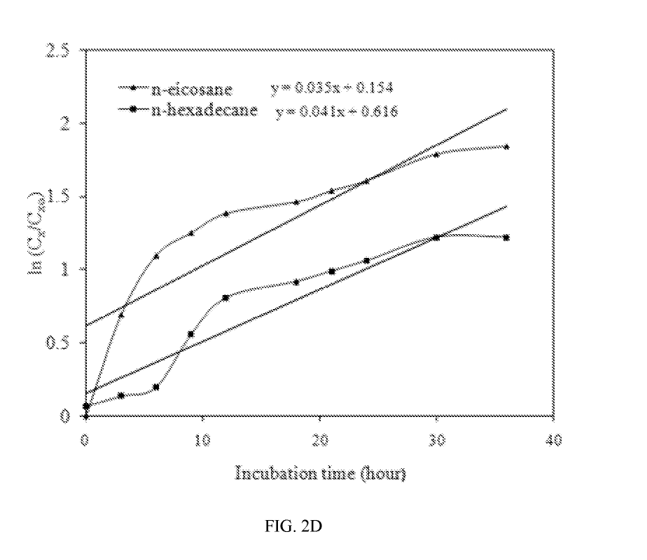

Pseudomonas aeruginosa showed rapid growth in the presence of n-hexadecane, as measured by CFU. Biomass dry weight increased at day 1, and then decreased linearly as the microbe attained log phase, and stabilized after attaining the dead phase (FIG. 2A). However, in the presence of n-eicosane, the growth was relatively slow at day 1, and decreased linearly after the microbes attained the log phase (FIG. 2A). The specific growth rate of Pseudomonas aeruginosa in the presence of n-eicosane and n-hexadecane was calculated to be 0.019 h⁻¹ and 0.05 h⁻¹, respectively, with doubling time of 36.48 h and 13.86 h (FIG. 2B). The growth of Pseudomonas fluorescens in the presence of n-eicosane and n-hexadecane increased. However, the growth continued up to 40 days in the presence of n-hexadecane (FIG. 2C). The specific growth rate of Pseudomonas fluorescence in the presence of n-eicosane and n-hexadecane was found to be 0.041 h⁻¹ and 0.035 h⁻¹, respectively, with doubling time of 16.9 hours and 19 hours (FIG. 2D).

### Example 2

### Emulsification and Reduction in Surface Tension of Paraffin Wax by Microbial Mixture

Microbes Pseudomonas aeruginosa and Pseudomonas fluorescens were grown in culture media as in Example 1, and the emulsification activity of the culture broth was measured as follows. An equal volume (2 mL) of kerosene and culture broth were mixed in a flat-bottomed test tube and mixed at high speed for 2 minutes using a vortex top mixer. After 24 hours, emulsification activity (EA) was calculated as follows:

$$EA = ( \frac{Height of the emulsion layer}{Height of the total mixture} ) \times 100$$

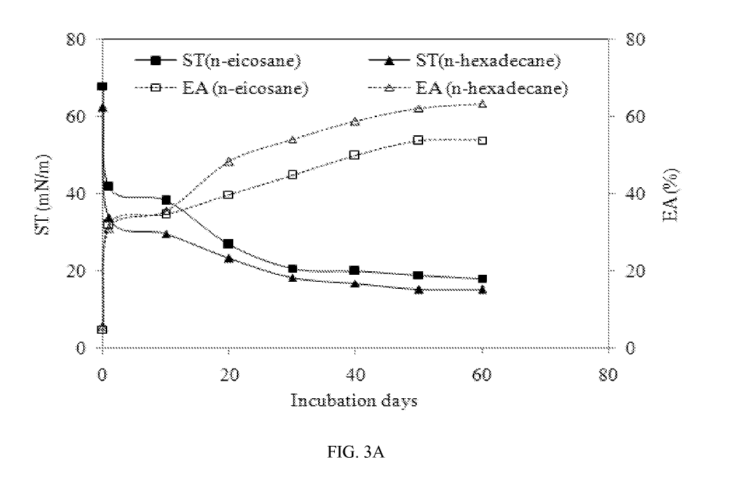

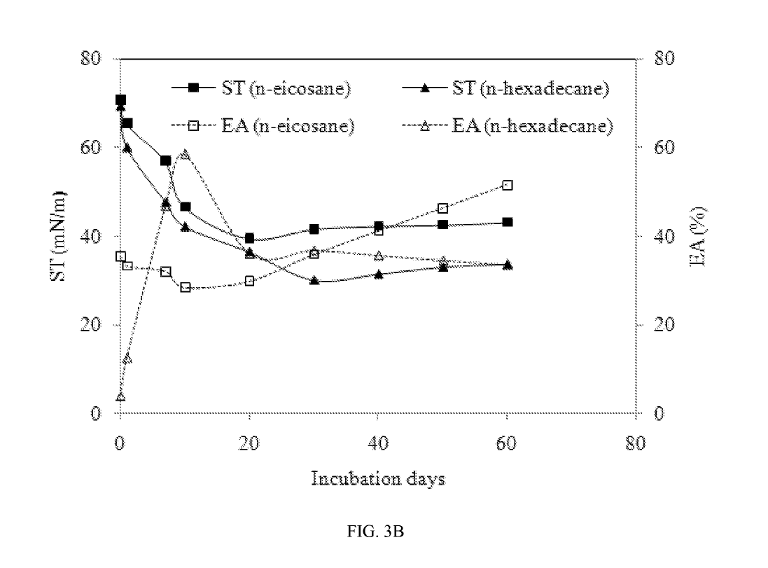

Emulsification activity of the culture broth from Pseudomonas aeruginosa grown in n-eicosane and n-hexadecane was found to be 53.9% and 63.42% respectively. For culture broth from Pseudomonas fluorescens grown in n-eicosane and n-hexadecane, the emulsification activity was found to be 35% and 50%, respectively (FIGS. 3A and 3B).

For measuring surface tension (ST), the culture broth was centrifuged at 4° C. for 10 min at 10000 rpm, and the cell-free supernatant was used. Surface tension was measured using a tensiometer (DAC11EA, Data physics) at 25° C. The platinum strip was dipped into the sample for attainment of equilibrium and the readings were obtained using the Static Contact Angle measuring device and Tensiometer (SCAT) software. For Pseudomonas aeruginosa, the surface tension of the broth decreased rapidly during the initial growth phase, and then decreased gradually and stabilized finally as the microbe entered the dead phase. The surface tension of n-eicosane broth reduced 73.48% from the initial value, and for n-hexadecane broth it reduced by 75.77% of the initial value. For Pseudomonas fluorescens, the surface tension of n-hexadecane broth reduced 57% in 40 days, but later increased slightly. This slight increase in surface tension may be due to the accumulation of microbial dead cells. The surface tension of n-eicosane broth reduced 41% in 30 days (FIGS. 3A and 3B).

### Example 3

### Reduction in Viscosity of Paraffin Wax by Microbial Mixture

Pseudomonas aeruginosa and Pseudomonas fluorescens were grown in culture media as in Example 1 and reduction in viscosity of the culture broth was measured as follows. Viscosity was measured using LV2T Brookfield viscometer using a small sample adapter of capacity 6.7 mL, with a spindle specification SC4-18. RheocalT software was used to calculate the viscosity. Viscosity was measured by varying the temperatures from 10° C. to 40° C., at the shear rate of 200 rpm.

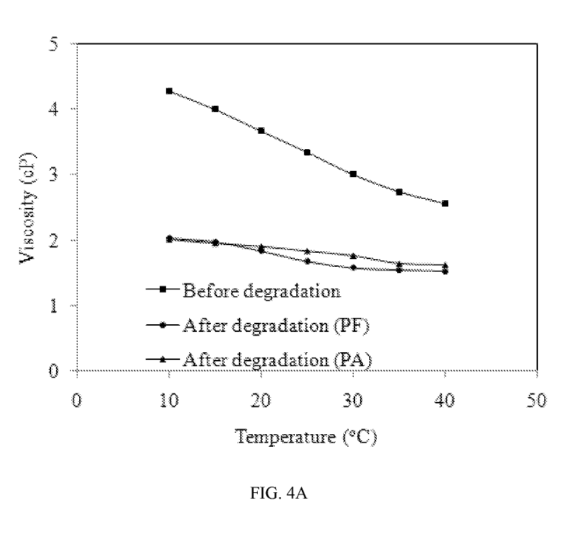

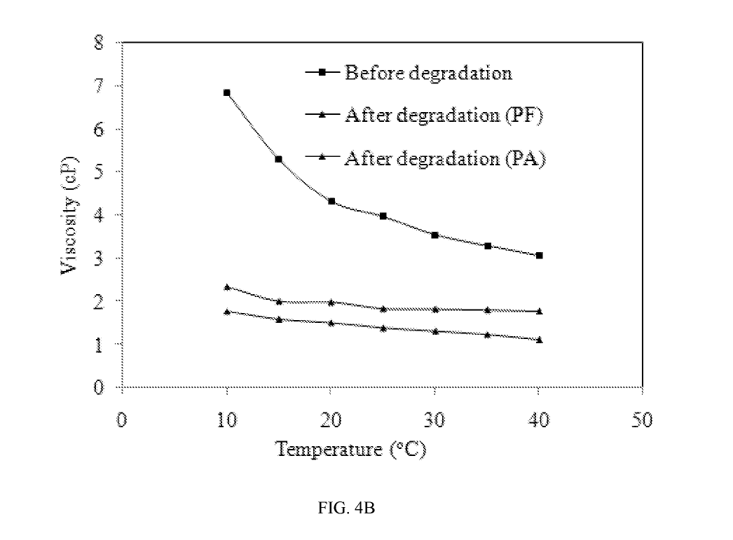

For Pseudomonas aeruginosa, the culture broth containing n-eicosane and n-hexadecane had a reduction in viscosity of 65% and 43%, respectively, at 25° C. The initial viscosity of n-hexadecane broth was 3.34 cP, and during the course of the experiment, it decreased to 1.83 cP. Similarly, the viscosity of n-eicosane decreased from 3.98 to 1.39 cP (FIG. 4A).

For Pseudomonas fluorescens, the culture broth containing n-eicosane and n-hexadecane had a reduction in viscosity of 54% and 50%, respectively (FIG. 4B).

### Example 4

### Degradation of Paraffin Wax by Microbial Culture

Microbes Pseudomonas aeruginosa and Pseudomonas fluorescens were grown in culture media as in Example 1 and the degradation of n-eicosane and n-hexadecane were monitored by GC-MS. The hydrocarbons from the culture supernatant were extracted using an equal amount of ethyl acetate, and evaporated using a rotary vacuum evaporator. The precipitate obtained was re-dissolved in ethyl acetate and filtered through 0.2 mm filter paper before analysis. The GC-MS was equipped with HP 5MS capillary column of medium polarity. The flow rate was set at 4 mL/min and the purge flow rate was 3 mL/minute. The injector and interface temperatures were kept at 220° C. and 250° C., respectively. Helium was used as the carrier gas at the flow rate of 1 mL/minute.

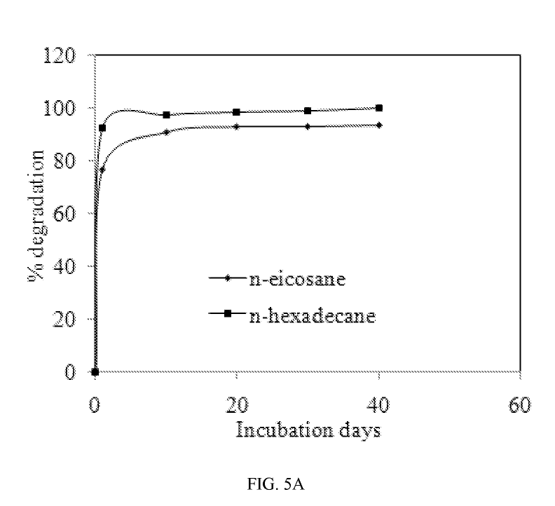

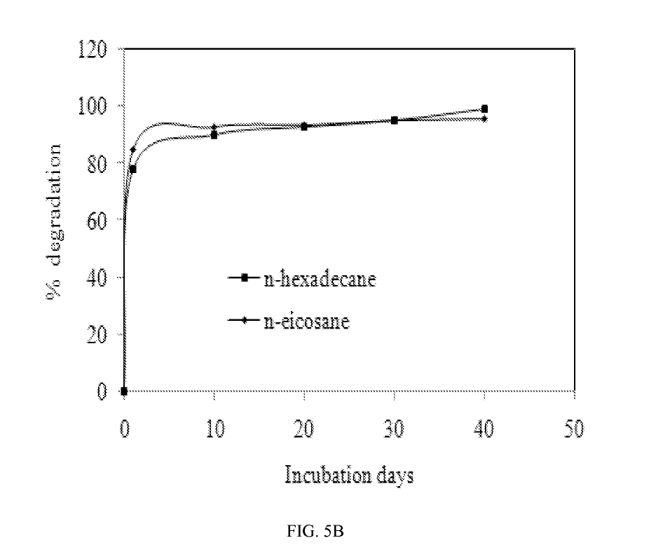

In case of Pseudomonas aeruginosa, about 93.9% of n-eicosane and about 99.8% of n-hexadecane were degraded in the culture broth in 40 days (FIG. 5). At day 1, about 92.52% of n-hexadecane and 76.77% of n-eicosane was degraded (Table 1). The degradation products of n-hexadecane were found to be 10-hydroxy-5,7-dimethoxy-2,3-dimethyl-1,4-anthracenedione, uline, chlorozotocin, 5,6,7,8,9,10-hexahydro-9-phenyl-spiro-2-thioneisoquinoline, agaricic acid, 2-heptadecanol acetate, 5-octyl methyl ester, and octanoic acid. N-eicosane degradation products were n-hexadecanoic acid, elaidic acid, isopropyl ester, di-n-octyl phthalate, 2,3, hydroxyl propyl ester, and 9-octadecanoic acid-2,3-dihydroxy propyl ester.

TABLE 1

| | |
|---|---|
| Incubation | % degradation |

| S. No | period (days) | n-hexadecane | n-eicosane |
|---|---|---|---|
| 1 | 0 | 0 | 0 |
| 2 | 1 | 92.52 | 76.77 |
| 3 | 10 | 97.44 | 90.81 |
| 4 | 20 | 98.73 | 92.87 |
| 5 | 30 | 99.05 | 93.21 |
| 6 | 40 | 99.89 | 93.88 |

Similarly, in the case of Pseudomonas fluorescens, about 96.8% of n-eicosane and about 99.4% n-hexadecane were degraded in 40 days (Table 2). Pseudomonas fluorescence degraded n-eicosane to trimethyl ester, octa siloxane, hexadecane, propanoic acid, trimethyl silyl ester, eicosamethyl, cyclodecasiloxane, and prosta-5,13-dien-1-oic acid. The degradation products of n-hexadecane were found to be 10-hydroxy-5,7-dimethoxy-2,3-dimethyl-1,4-anthracenedione, uline, chlorozotocin, 5,6,7,8,9,10-hexahydro-9-phenyl-spiro-2-thione-isoquinoline, agaricic acid, 2-heptadecanol acetate, 5-octyl methyl ester, and octanoic acid.

TABLE 2

| | |
|---|---|
| Incubation | % degradation |

| S. No | period (days) | n-hexadecane | n-eicosane |
|---|---|---|---|
| 1 | 0 | 0 | 0 |
| 2 | 1 | 78 | 85 |
| 3 | 10 | 90.21 | 92.9 |
| 4 | 20 | 92.87 | 93.6 |
| 5 | 30 | 94.98 | 95 |
| 6 | 40 | 99 | 96 |

### Example 5

### Degradation of Oil Sludge in a Wellbore Tubing

A starter culture of microbial mixture containing nutrient medium is grown as in Example 1. A batch culture of 1000 L is prepared and injected into a wellbore tubing under regulated pressure, using a slow injection rate (10 L/minute). The well is then closed for a period of time (for example, 7 days) sufficient for development of a biofilm on the inner surface of wellbore tubing. The biofilm formed will cause dissociation and degradation of the wax particles in the wellbore tubing. Upon resuming oil flow, the biofilm prevents wax deposition in the wellbore tubing. Thus, the presence of the biofilm provides enhanced oil flow though the wellbore tubing and prevents further constriction of the pipe by wax deposits in the wellbore tubing.

### Example 6

### Degradation of Tank Bottom Sludge

A starter culture of microbial mixture containing nutrient medium is grown as in Example 1. A batch culture of 1000 L is prepared and introduced into an oil storage tank. The tank is closed for a period of 4 weeks to allow growth of microbes. The microbes degrade high molecular weight hydrocarbons present in the sludge. Samples of the sludge are withdrawn periodically to monitor the degradation. The microbes cause the sludge to become mobile and the sludge is easily removed by suction. The tank is ready for storing oil.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of degrading an oil sludge in a pipeline, the method comprising:
introducing a microbial mixture comprising a Pseudomonas sp. and a nutrient medium into the pipeline such that the microbial mixture contacts the oil sludge, wherein the Pseudomonas sp. is selected from the group consisting of Pseudomonas aeruginosa, Pseudomonas fluorescens, and any combination thereof.

2-3. (canceled)

4. The method of claim 1, wherein introducing the microbial mixture comprises introducing a microbial mixture comprising the Pseudomonas sp. and the nutrient medium, wherein the nutrient medium comprises KH₂PO₄, Na₂HPO₄, NH₄Cl, NaCl, glucose, MgSO₄, or any combination thereof.

5. The method of claim 1, wherein introducing the microbial mixture comprises introducing a microbial mixture comprising the Pseudomonas sp. and the nutrient medium, wherein the nutrient medium comprises about 0.1 wt. % to about 0.4 wt. % KH₂PO₄, about 0.3 wt. % to about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

6. (canceled)

7. The method of claim 1, wherein introducing the microbial mixture results in degradation of about 70% to about 99% of the oil sludge to a lower alkane, an ester, a primary alcohol, an aldehyde, a fatty acid, a dicarboxylic acid, acetyl CoA, or any combination thereof in a period of about 1 day to about 3 days.

8. The method of claim 1, wherein introducing the microbial mixture comprises introducing a microbial mixture further comprising at least one surfactant, wherein the surfactant is stable up to a temperature of about 100° C. and at a pH of about 2 to about 14.

9-10. (canceled)

11. The method of claim 1, wherein the microbial mixture having an emulsification index of about 30% to about 70% is introduced.

12-13. (canceled)

14. The method of claim 1, wherein the microbial mixture when in contact with the oil sludge for a sufficient period of time decreases a viscosity of the oil sludge by about 30% to about 60% at below wax appearance temperature (WAT) and promotes flow assurance in the pipeline.

15-17. (canceled)

18. A pipeline comprising an oil sludge, a microbial mixture comprising a Pseudomonas sp. and a nutrient medium disposed within the pipeline, wherein the microbial mixture is in contact with the oil sludge, and wherein the Pseudomonas sp. is selected from the group consisting of Pseudomonas aeruginosa, Pseudomonas fluorescens, and any combination thereof.

19. (canceled)

20. The pipeline of claim 18, wherein the nutrient medium comprises KH₂PO₄, Na₂HPO₄, NH₄Cl, NaCl, glucose, MgSO₄, or any combination thereof.

21. The pipeline of claim 18, wherein the nutrient medium comprises about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

22-24. (canceled)

25. A biofilm comprising a Pseudomonas sp. and a nutrient medium, wherein the nutrient medium comprises KH₂PO₄, Na₂HPO₄, NH₄Cl, NaCl, glucose, MgSO₄, or any combination thereof.

26. The biofilm of claim 25, wherein the nutrient medium comprises about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

27. The biofilm of claim 25, wherein the Pseudomonas sp. is Pseudomonas aeruginosa, Pseudomonas fluorescens, or any combination thereof.

28. The biofilm of claim 25, wherein the biofilm further comprises a polysaccharide matrix in contact with the Pseudomonas sp. and the nutrient medium.

29-34. (canceled)

35. A method of biodegrading an oil sludge, the method comprising:
contacting the oil sludge with a microbial mixture comprising a Pseudomonas sp. and a nutrient medium, wherein about 70% to about 99% of the oil sludge is degraded in a period of about 1 day to about 3 days, and wherein the Pseudomonas sp. is selected from the group consisting of Pseudomonas aeruginosa, Pseudomonas fluorescens, and any combination thereof.

36-37. (canceled)

38. The method of claim 35, wherein the oil sludge is contacted with the microbial mixture comprising the Pseudomonas sp. and the nutrient medium, wherein the nutrient medium comprises KH₂PO₄, Na₂HPO₄, NH₄Cl, NaCl, glucose, MgSO₄, or any combination thereof.

39. The method of claim 35, wherein the oil sludge is contacted with the microbial mixture comprising the Pseudomonas sp. and the nutrient medium, wherein the nutrient medium comprises about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

40. The method of claim 35, wherein the oil sludge is contacted with the microbial mixture, wherein the microbial mixture further comprises at least one surfactant, and the surfactant is stable up to a temperature of about 100° C. and stable at a pH of about 2 to about 14.

41-42. (canceled)

43. The method of claim 40, wherein the oil sludge is contacted with the microbial mixture comprising the at least one surfactant secreted by the Pseudomonas sp.

44-45. (canceled)

46. The method of claim 35, wherein the oil sludge is contacted with the microbial mixture, wherein the microbial mixture has an emulsification index of about 30% to about 70%.

47. (canceled)

48. The method of claim 35, wherein the oil sludge is contacted with microbial mixture for about 1 day to about 60 days at a temperature of about 5° C. to about 50° C.

49-58. (canceled)

59. A method of preventing a build-up of an oil sludge in a pipeline, the method comprising:
providing an oil flow in the pipeline; and
introducing a microbial mixture comprising a Pseudomonas sp. and a nutrient medium into the pipeline, wherein the Pseudomonas sp. is selected from the group consisting of Pseudomonas aeruginosa, Pseudomonas fluorescens, and any combination thereof.

60-61. (canceled)

62. The method of claim 59, wherein introducing the microbial mixture comprises introducing a microbial mixture comprising the Pseudomonas sp. and a nutrient medium, wherein the nutrient medium comprises about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.

63. (canceled)

64. A method of pretreating crude oil, the method comprising:
contacting crude oil with a microbial mixture comprising a Pseudomonas sp. and a nutrient medium, wherein the microbial mixture degrades about 70% to about 99% of an oil sludge present in the crude oil in a period of about 1 day to about 3 days, and wherein the Pseudomonas sp. is selected from the group consisting of Pseudomonas aeruginosa, Pseudomonas fluorescens, and any combination thereof.

65-66. (canceled)

67. The method of claim 64, wherein the crude oil is contacted with the microbial mixture comprising the Pseudomonas sp. and the nutrient medium, wherein the nutrient medium comprises about 0.1 wt. % to about 0.4 wt. % KH₂PO₄, about 0.3 wt. % about 0.8 wt. % Na₂HPO₄, about 0.1 wt. % to about 0.3 wt. % NH₄Cl, about 0.3 wt. % to about 0.7 wt. % NaCl, about 0.5 wt. % to about 1 wt. % glucose, about 0.01 wt. % to about 0.05 wt. % MgSO₄, or any combination thereof.
